(19) United States
(12) Patent Application Publication
(10) Pub. No.: US 2026/0027419 A1
(43) Pub. Date: Jan. 29, 2026

(54) VESTIBULAR TRAINING APPARATUS WITH GUARD RAILING AND METHOD OF USE

(71) Applicants: Sheila Thelen, Shafer, MN (US); Jonathan Wampler, Middleton, DE (US); Robert Briccotto, Middleton, DE (US)

(72) Inventors: Sheila Thelen, Shafer, MN (US); Jonathan Wampler, Middleton, DE (US); Robert Briccotto, Middleton, DE (US)

(21) Appl. No.: 18/785,809

(22) Filed: Jul. 26, 2024

(51) Int. Cl. A63B 26/00; A61B 5/00

(52) U.S. Cl. CPC A63B 26/003; A61B 5/4023

(57) ABSTRACT

A vestibular training apparatus and method as disclosed and claimed. Wherein the training apparatus comprises a motorized rotational platform. a waist harness, a plurality of balusters, a guard rail, a guard rail aperture, and a plurality of straps.

### TECHNICAL FIELD

The present disclosure relates to a vestibular training apparatus with guard railings and a method of use. Vestibular training is beneficial to increase coordination, balance, dual hemispheric brain function, and cognition in users. In addition, vestibular training is helpful in the treatment of autism, mild traumatic brain injury (mTBI), traumatic brain injury (TBI), and post-concussion therapy.

### BACKGROUND

Devices designed to enhance dual hemispheric left and right brain connectivity are beneficial in general to cognitive health. Left and right brain connectivity are enhanced by many therapeutic and brain training techniques, one of which is vestibular training.

In particular, individuals with autism, mTBI, TBI and concussions have been shown to benefit from vestibular training and therapy. The method implements a type of rotational motion where limbs cross the medial axis. In addition, individuals that perform fast rotation sports which require a high degree of balance and coordination, such as figure skating, may also benefit from vestibular training exercises.

Development of the central nervous system, specifically vestibular training is beneficial to cognition, perceptual motor, and sensory motor skills. Specifically, vestibular training is beneficial to the development of skills such as auditory learning, visual perception, attention, and focus. In addition, vestibular training benefits, hand-eye coordination, ocular motor control, and postural adjustment. Vestibular training also benefits tactile and proprioception parts of the central nervous system.

The object of the invention is a vestibular training apparatus and method utilized for enhancement of cognitive function. Such an apparatus and method may be utilized by an individual with a disorder such as autism, mTBI, TBI, post-concussion, or any individual seeking to enhance their cognitive function.

### SUMMARY

The present disclosure describes a vestibular training method and apparatus. The apparatus and method both comprise a motorized rotational platform [8], a guard railing [2], supported by balusters [4], an adjustable waist harness [1] worn by a user [3]. Said waist harness [1] is connectively coupled to the balusters [4].

In use the waist harness [1] is adjusted to the size of the user [3] and connectively coupled to the balusters [4] by one or more straps [5]. In use, a motorized rotational platform [8] will rotate a user [3] at variable speeds.

The apparatus may also be utilized with a pressure sensor [9] wherein the user will stand on the weight pressure sensor prior to utilizing the apparatus, and after. The benefits to balance and posture may therefore be observed and tracked following vestibular training.

### BRIEF DESCRIPTION OF DRAWINGS

FIG. 1 is a slightly elevated perspective view of a vestibular training apparatus with a user disposed therein and trainer manipulating the device.

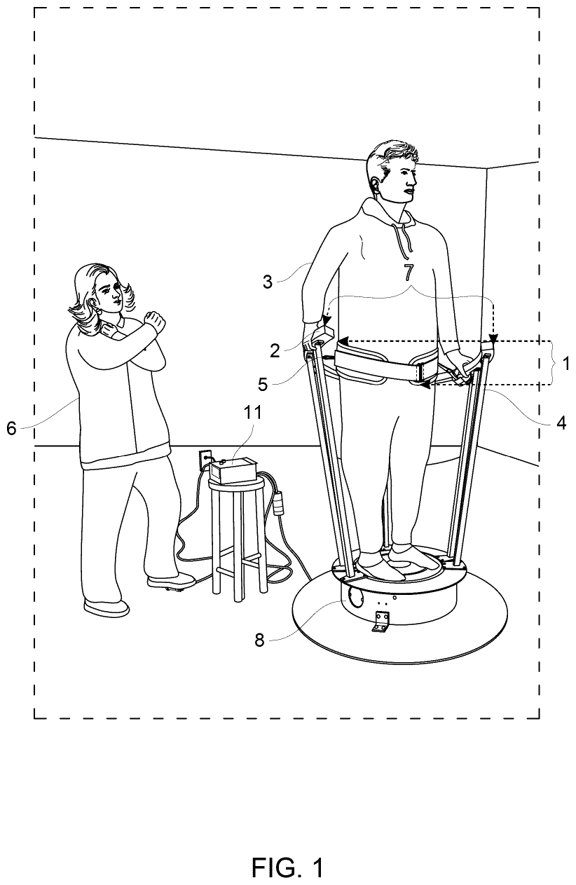

FIG. 2 is a perspective view of the vestibular training apparatus without a user.

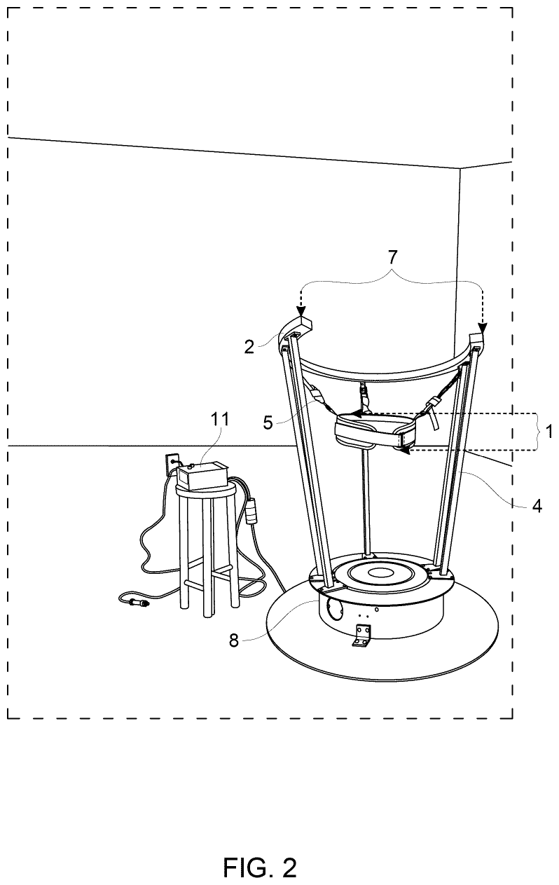

FIG. 3 is a perspective view of vestibular training apparatus with a pressure sensor and computer.

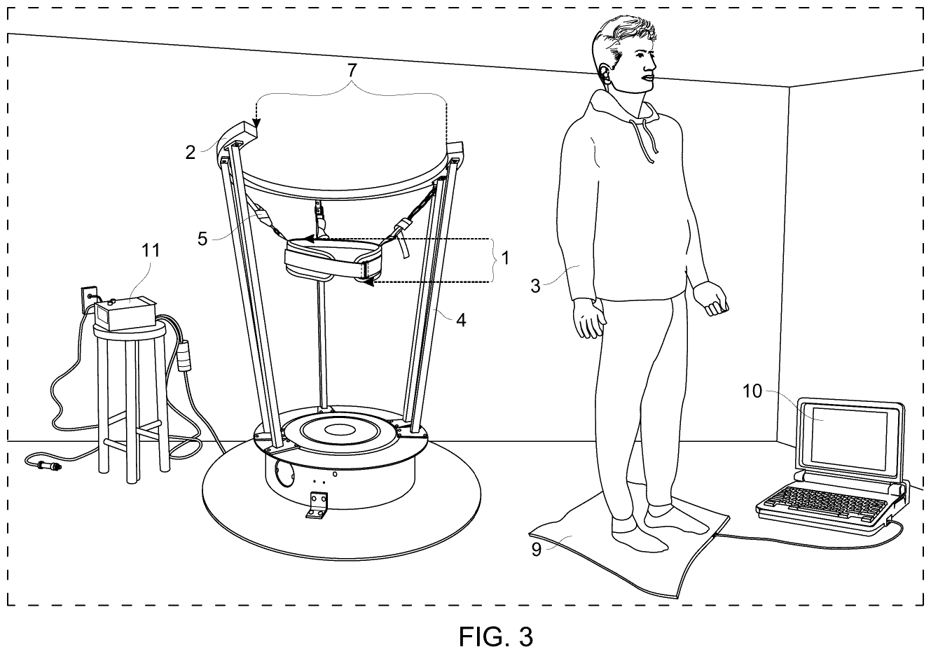

FIG. 4 is a flow diagram illustrating an example method of vestibular training.

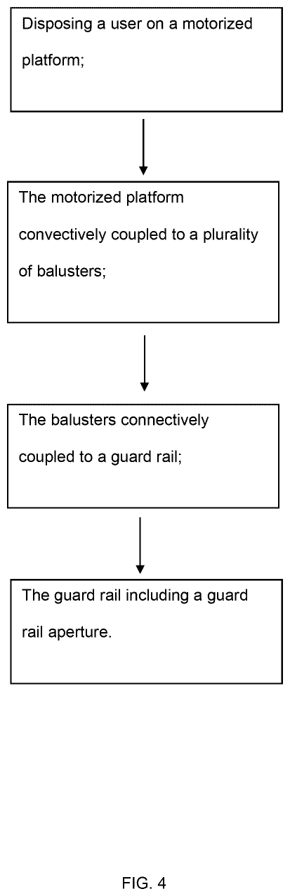

FIG. 5 is a flow diagram illustrating an example method of vestibular training with a pressure sensing device.

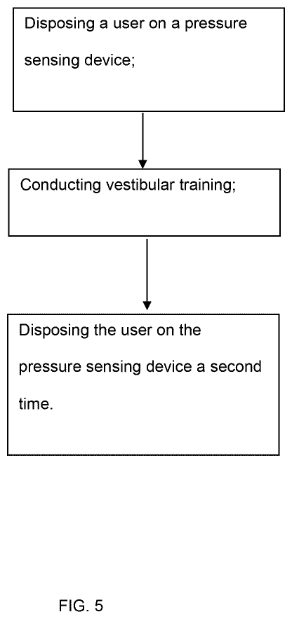

### DETAILED DESCRIPTION

The present disclosure describes a vestibular training apparatus and method of use.

An apparatus is disclosed. Said apparatus comprises a motorized rotational platform [8], a waist harness [1], balusters [4], straps [5] and a guard railing [2].

In one embodiment, a user [3] is disposed on the motorized rotational platform [8], a waist harness [1] is worn on the waist of the user [3]. Said waist harness [1] connected to the balusters [4] via straps [5]. The motorized rotational platform [8] rotates the user [3].

The guard railing [2] includes a guard railing aperture [3] configured to allow the user [3] to enter and exit the interior of the guard railing [2]. Said rotational platform [8] rotates with the user [3] standing on the motorized rotational platform [8]. In motion, the straps [5] support the user [3] and stop him or her from falling.

To use the apparatus and practice the method, the user [3] will stand on a motorized rotational platform [8]. Various commercial rotational platforms are available such as those offered by R&J Spinners. In one embodiment, the motorized rotational platform [1] will rotate, in a velocity set by a trainer [6] or user [3]. The motorized rotational platform [1] has a series of settings such that the user [3] may be rotated greater or fewer rotations per minute. A higher frequency of rotation may be used where a user [3] has utilized the apparatus and method more often. The rotation frequency and direction is controlled by a platform control panel [11].

Said motorized rotational platform [8] must be of requisite size to accommodate the length of a foot for an adult or youth person. Also, the motorized rotational platform [8] must be of requisite power to rotate an adult or a youth ranging from 20 to 400 pounds or more.

In one embodiment, a user [3] will stand on the motorized rotational platform [8] for a period of one to several seconds and be rotated along with the rotational platform around a vertical axis.

In one embodiment, said harness [1] comprises nylon webbing material. The harness is attached to the balusters [4] via straps [5]. In one embodiment, there are three straps connecting the harness [1] to three balusters [4]. In this embodiment, there are a total of five balusters [4].

In one embodiment, a pressure sensor [9] may be utilized with the apparatus. The user [3] will take measurements on the pressure sensor [9] prior to utilizing the apparatus. Following a training session, the user [3] will again stand on pressure sensor to track their improvement in such factors as posture, balance, equilibrium, and weight distribution.

In one embodiment, the pressure sensor [9] is configured to measure the weight distribution, posture, equilibrium, and balance of the individual. The benefits in the weight distribution, posture, equilibrium, and balance of the individual following vestibular training are then trackable.

In one embodiment, the measurements from the pressure sensor [9] will be maintained and tracked over the course of many vestibular training sessions. Thus, the benefits to the user [3] will be measured and recorded. By utilizing a series of measurements, the training sessions may be modified to enhance certain treatment objectives that the user [3] is seeking to improve.

The pressure sensor measurement device may be a variety of devices that track posture, weight distribution, equilibrium, stability or balance. One such manufacturer is the Boditrack by Vista Medical.

In one embodiment, the pressure sensor [9] will be connected to a computer that can record the measurements taken by the sensor. Said computer is configured to record and prepare reports on the measurements taken by the sensor, such as graphical representations of data and trends of the measurements taken.

1. A vestibular training apparatus comprising:
a motorized platform;
a waist harness;
a plurality of balusters;
a guard rail;
a guard rail aperture; and
a plurality of straps.

2. The vestibular training apparatus of claim 1, wherein a user is disposed within the waist harness.

3. A vestibular training apparatus of claim 1, wherein the plurality of straps couple the waist harness to the plurality of balusters.

4. The vestibular training apparatus of claim 1, wherein the motorized platform is configured to rotate 360 degrees.

5. A method of conducting vestibular training comprising;
disposing a user on a motorized platform;
the motorized platform convectively coupled to a plurality of balusters;
the balusters connectively coupled to a guard rail;
the guard rail including a guard rail aperture.

6. A method of claim 5, wherein the user is rotated by the motorized platform.

7. A method of claim 5, wherein the motorized platform includes a variety of settings such that the speed and direction of rotation may be varied.

8. A method of claim 5, wherein the user is disposed within a waist harness.

9. A method of claim 5, wherein the waist harness is coupled to the plurality of balusters via a plurality of straps.

10. A method of claim 5, wherein the platform rotates the user 360 degrees.

11. A method of claim 5, wherein the user is an individual with a disorder from the set of, autism, traumatic brain injury, mild traumatic brain injury, or concussion.

12. A method of claim 5, wherein the user is conducting training for figure skating.

13. A method of claims 5, wherein the user is conducting training for activities incorporating rotation.

14. A method of conducting vestibular training comprising;
disposing a user on a pressure sensing device;
conducting vestibular training;
disposing the user on the pressure sensing device a second time.

15. A method of claim 14, wherein the pressure sensing device will record measurements over the course of a plurality of vestibular training sessions.

16. A method of claim 14, wherein a motorized platform is rotated at a certain speed based on the results of measurements taken from the pressure sensor.

17. A method of claim 14, wherein the direction of rotation of the motorized platform is set based on the results of measurements taken from the pressure sensor.

18. A method of claim 14, wherein vestibular training takes place at different time intervals based the measurements from the pressure sensor.

19. A method of claim 14, wherein the measurements from the pressure training sensor direct the course of treatment for a neurological condition.

20. A method of claim 14, wherein the measurements from the pressure training sensor are recorded and analyzed to track the treatment for a neurological condition.
